(19) United States
(12) Patent Application Publication
(10) Pub. No.: US 2020/0315802 A1
(43) Pub. Date: Oct. 8, 2020

(54) Additive Manufacture Of Medical Implants And Implants So Manufactured

(71) Applicant: DiFusion, Inc., Austin, TX (US)

(72) Inventors: Joseph J. Crudden, Hudson, NH (US); Derrick Johns, Austin, TX (US); Sriram Sankar, Austin, TX (US)

(21) Appl. No.: 16/905,110

(22) Filed: Jun. 18, 2020

Related U.S. Application Data
(63) Continuation of application No. 16/369,147, filed on Mar. 29, 2019.
(60) Provisional application No. 62/649,844, filed on Mar. 29, 2018.

(51) Int. Cl. A61F 2/28; A61L 27/34; A61L 27/56; A61F 2/00

(52) U.S. Cl. CPC A61F 2/28; A61L 27/34; A61F 2/32; A61F 2/0077; A61L 27/56

(57) ABSTRACT

Anti-biofilm osseointegrating implantable devices are made by additive manufacturing. A powder formulation is made that includes a resin such as a polyarylether ketone such as PEEK, and a zeolite, and the zeolite may be loaded with one or more therapeutic metal ions, such as silver, copper and/or zinc that exhibit antimicrobial properties. The powder formulation also may include a porogen to control the porosity of the resulting three-dimensional implant device. The devices, which are osseointegrating, may include metal-loaded zeolite so as to elute antimicrobial metal ions in a therapeutically effective amount when implanted into a body and exposed to bodily fluid.

This application is a continuation of U.S. patent application Ser. No. 16/369,147 filed Mar. 29, 2019, which claims priority of U.S. Provisional Application Ser. No. 62/649,844 filed Mar. 29, 2018, the disclosures of which are incorporated herein by reference in their entireties.

### BACKGROUND

Implantable medical devices are surgically implanted into the body for various reasons, including orthopedic applications (e.g., hip replacement, skull flaps, dental implants, spinal procedures, knee replacement, bone fracture repair, etc.). In view of the structural integrity required by many such devices, materials of fabrication are limited and generally consist of metal, plastic and composites.

The benefits derived from these devices are often offset by infection which in some cases can lead to sepsis and death. The most common organisms causing infections are Staphylococcus epidermidis and Staphylococcus aureus. Staphylococcus epidermidis is a major component of the normal bacterial flora of human skin and mucous membranes. It is a common pathogen that often colonizes patients in hospital settings who have surgical implants due to the microbes' ability to adhere to medical devices and form a biofilm. Additionally, methicillin-resistant Staphylococcus aureus (MRSA) is a type of Staphylococcus bacteria that is resistant to many antibiotics is therefore of particular concern. Other gram-positive bacteria, gram-negative bacteria and fungal organisms also are causative organisms that may be problematic.

As microorganisms come in close proximity to the surface of the medical device, they will either be attracted or repelled by it depending on the sum of the different non-specific interactions. In biological systems, hydrophobic/hydrophilic interactions play an important role in the pathogenesis of a wide range of microbial infections.

Thermoplastic resins, including polyetherketoneketone (PEKK) and polyetheretherketone (PEEK) have been found to be a useful material for medical implants. PEEK is particularly suitable because its modulus of elasticity closely matches that of bone. It is also radiotranslucent. However, PEEK is a hydrophobic material, very resistant to permeation by liquids, and bacteria tend to adhere easily to these types of surfaces. It is also an organic material which does not carry significant surface charges. PEEK does not interact well with tissue, nor does it osseointegrate with bone. Indeed, PEEK implants present a smooth hydrophobic, uncharged, inert surface to surrounding tissue. These surfaces are not recognized as natural and become encapsulated by a fibrous apposition layer of soft tissue rather than becoming bonded to bone and tissue cells.

As a result, zeolite has been incorporated into PEEK to create a composite material with ceramic character that confers charge to the surface and renders it hydrophilic. Ceramics such as zeolite function as a cation cage, being able to be loaded with silver and other cations having antimicrobial properties. Metal zeolites can be used as an antimicrobial agent, such as by being mixed with the resins used as thermoplastic materials to make the implantable devices, or coatings to be applied to the devices. The antimicrobial metal zeolites can be prepared by replacing all or part of the ion-exchangeable ions in zeolite with ammonium ions and antimicrobial metal ions. Such materials have been seen to perform extremely well in ovine and rabbit implant studies, showing high tissue compatibility, and bonding very well to bone and soft tissues alike.

Hip and knee implants have been very successful and provided a new lease on life for otherwise incapacitated patients. However, patients who have received metal hip and knee implants, particularly patients treated after tumor rescission, have been at risk of infection, aseptic loosening and in the worst cases, may need to have the limb amputated. Recent studies have shown that silver eluting hip stems can significantly improve outcomes, essentially eliminating the need for amputations, However, poorly controlled release of silver because of device design can result in the deleterious accumulation of excess silver in the joint over time. Release of silver and other therapeutic metal ions, from ion exchange ceramics such as zeolites, incorporated into polymer composites which are used to fabricate orthopedic devices can provide for precision controlled release of the correct, safe and efficacious level of therapeutic ion.

Trauma plates and other forms of hardware are often used to repair broken bones, etc. If even a few bacteria attach to the surface of the repair device, lack of union, deep infection and even a failure of the surgical wound to close can ensue. Sometimes it may be necessary to transfer tissue such as muscle from another site, to bridge the surgical wound. Subsequently there may still be problems with recurrent infections from biofilm on the device, especially if an antibiotic resistant strain develops. Many repeat surgeries may be necessary, reducing the chance for a positive outcome.

One of the most common incision sites in bone cancer surgeries is at the end of the femur, close to the knee joint. To completely remove the tumor and reconstruct a functioning extremity, accuracy of an incision site is critical. Correcting pelvic fractures from falls or other accidents is another routine surgical procedure that requires extreme precision. Indeed, there are numerous complex surgical procedures that require, or would greatly benefit from, precisely manufactured medical devices, including angle of attachment points (e.g., screw placement and location) in medical implants.

Additive manufacturing, such as 3D printing, has become commonplace in recent years. In contrast to subtractive manufacturing, which involves removal of material from a blank block using equipment such as lathes, milling machines and drill to reveal the desired structured object, additive manufacturing builds up the object by adding material by extrusion or laser sintering using precise computer control using a pre-generated computer design.

Accordingly, it would be desirable to provide a precise manufacturing method for making engineered implantable medical devices in order to customize the implant to the particular patient and/or surgery. It also would be desirable to precisely manufacture such devices with the inclusion of antimicrobial agents that function to reduce the growth of bacteria and risk of infection, and that exhibits hydrophilicity and a negative charge so as to promote osseointegration.

### SUMMARY

The shortcomings of the prior art have been overcome by embodiments disclosed herein, which relate to additive manufacturing methods for making engineered anti-biofilm osseointegrating implantable biomaterial devices that optionally can elute therapeutic ions such as silver. In certain embodiments, medical devices such as implants are engineered prepared by additive manufacture such as 3D printing to produce a 3D structure suitable as an implant. The resulting device may have the form of the desired implant, e.g., a hip stem, skull flap, spinal implant (e.g., an intervertebral spacer), dental implant, screw, rod, hip stem, spinal spacer, skull flap or trauma plate. In certain embodiments, the implants are orthopedic implants, such as spinal, knee and hip implants, and are so shaped or configured. In some embodiments, the polymer includes a polyarylether ketone such as polyetheretherketone (PEEK). In some embodiments, the polymer also may include zeolite, and the zeolite optionally may be loaded with one or more therapeutic metal ions, such as silver, copper and/or zinc that exhibit antimicrobial properties when implanted into a body and exposed to bodily fluid or tissue. The devices, when implanted into a body and exposed to bodily fluid, may elute antimicrobial metal ions in a therapeutically effective amount. In certain embodiments, the source of antimicrobial activity includes ion-exchangeable cations contained in a zeolite. In certain embodiments, disclosed are methods of imparting antimicrobial activity to devices by controlling the delivery of certain cations through ion-exchange via a zeolite incorporated in the device introduced in a patient.

In some embodiments, the zeolite does not contain an antimicrobial metal ion, yet imparts hydrophilicity and a negative charge to the implant. This helps prevent biofilm formation and enhances osseointegration. In embodiments where antimicrobial ions are present, the PEEK/zeolite combination increases the ability of antimicrobial moieties to permeate in and kill the bacterial pathogen rather than be repelled by the hydrophobic surface properties of naked PEEK.

In certain embodiments, the device is configured for use in spinal fusion (arthrodesis) which is often employed to stabilize an unstable spinal column due to structural deformity, trauma, degeneration, etc. Fusion is a surgical technique in which one or more vertebrae of the spine are united together (“fused”) to reduce or eliminate relative motion between them or to fix the spatial relationship between them. Spinal fusions include posterolateral fusion, posterior lumbar interbody fusion, anterior lumbar interbody fusion, anterior/posterior spinal fusion, cervical fusion, thoracic fusion and interlaminar fusion. In certain embodiments, the devices are for insertion in an intervertebral space between adjacent vertebrae. In certain embodiments, a fusion site is identified between adjacent vertebrae and a bone graft is implanted at said site. In certain embodiments, the implant is a spinal interbody cage, including cages comprising titanium, carbon fibers, biocompatible materials such as polyetheretherketone (PEEK), polyetherketoneketone (PEKK), or other synthetic substances. In certain embodiments, zeolite particles are incorporated into the PEEK interbody cage. In certain embodiments, the cage is loaded with osseoconductive and/or osseoinductive agents to promote fusion. Preferably, the implant includes PEEK resin, and ceramic particles are incorporated into the resin such that a negative charge is imparted to an exposed surface of the resin. The term “exposed surface” is intended to include one or more surfaces of an implantable device that when implanted, is exposed to or in contact with body tissue and/or fluids.

The hydrophilicity imparted by the zeolite results in an engineered biomaterial that interacts with the bone of the patient and induces a bone/biomaterial fusion. The presence of the zeolite also results in a rapid transition from M1 proinflammatory macrophage phenotype to the M2 macrophage phenotype, thereby minimizing fibrous encapsulation and facilitating the deposition of cite appropriate tissue ultimately yielding constructive and functional tissue remodeling. The negative charge imparted by the zeolite attracts and adheres the required precursor proteins for bone growth to the implant surface, and ultimately supports long term osseointegration.

### DETAILED DESCRIPTION

A more complete understanding of the components, processes, systems and apparatuses disclosed herein can be obtained by reference to the accompanying drawings. The figures are merely schematic representations based on convenience and the ease of demonstrating the present disclosure, and are, therefore, not necessarily intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2 inches to 10 inches” is inclusive of the endpoints, 2 inches and 10 inches, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, and “outward” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

Certain embodiments relate to a biomaterial formulated by blending a base polymer, preferably PEEK, with a negatively charged zeolite, and additive manufacturing an implant composite biomaterial using the blend. The zeolite changes the surface topography, charging characteristics, and pH of the resulting composite in a predictable, suitable manner for the surgical environment and long-term healing of the patient into which the device is implanted. Attributes imparted by the zeolite include bone fusion, biocompatibility, negative charge, hydrophilicity and osseoconductivity. Attributes provided by the PEEK base polymer include radiolucency, biocompatibility, durability and versatility. The resulting composite blend provides a uniform material construct and excellent workability.

Particularly compelling is the ability of the zeolite to reduce or eliminate the immune response that is generated when naked PEEK is implanted. It is a well-recognized problem that the human immune system reacts to the presence of naked PEEK as a foreign, unnatural substance, and as a damage/danger associated molecular pattern (DAMP). Consequently, the human body responds to the presence of naked PEEK by encapsulating it, causing bone resorption, and initiating a pain response. This is believed to be directly related to the hydrophobic, uncharged and water repellant nature of naked PEEK. Adding zeolite to the PEEK polymer increases proliferation, differentiation and transforms growth factor beta production in normal adult human osteoblast-like cells. The hydrophilic surface of the resulting implant down-regulates proinflammatory cytokines interleukin 1 & 6, which modulates the immune response, facilitates the enhanced bone would healing and osseointegration, allows for early cell adhesion and ultimate osteoconduction, and reduces pain. IL1-Beta upregulates inflammatory immune-response, and IL6-Beta haw been shown to have a direct relation to spinal disc pain. Both have been shown to down regulate osteoblast cells while up-regulating osteoclast cells, showing the increased fibrosis and resorption of bone with which naked hydrophobic PEEK has been well associated.

Accordingly, in certain embodiments, medical implants are manufactured using high temperature laser sintering, such as with an EOSINT P 800 system commercially available from EOS of North America Inc. In other embodiments, additive manufacturing of medical implants is carried out using a filament based, extrusion technique. For example, a zeolite PEEK composite filament (similar to the filament which is produced in the course of producing pellets for extrusion of rod) is made, and this filament may be used for extrusion printing or to make finer filaments for extrusion printing. The additive manufacturing process allows for concurrent deposition of the zeolite and the resin to form the implant.

The preferred method of 3D printing implants from high melting plastics such as PEEK and PEKK is by laser sintering in view of its precision. Other suitable resins include low density polyethylene, polypropylene, ultra-high molecular weight polyethylene or polystyrene, polyvinyl chloride, ABS resins, silicones, rubber, and mixtures thereof, and reinforced resins, such as ceramic or carbon fiber-reinforced resins, particularly carbon fiber-reinforced PEEK. PEEK is particularly preferred, and melts at between 385 and 400 degrees Celsius. Laser sintering functions by heating the plastic in powder form to just below the melting point and then uses a laser to add additional heat to liquefy the powder at precisely defined locations. One layer of the object is fused and then another layer of powder is added and fused in the exact areas defined by the CAD data. In this way, a 3D object can be built up, layer by layer. In certain embodiment, the resulting implants are load-bearing surgical implants. Those skilled in the art know how to convert data from a CT or MRI scan or CAD into a 3d printable model.

An alternative method of 3D printing is by extrusion of a bead or ribbon of the plastic composite through a heated nozzle, similar to a hot glue gun under computer control driven by CAD/CAM data. This process is less expensive but less precise than SLS, however, it works adequately for many applications. To use this process to produce the devices from the polymer composites, a ribbon of the composite is produced by extruding a fine thread of the material such as by using a heated twin screw extruder. A suitable extruder is commercially available from Leistritz. This process is also used to produce filaments which are cut into pellets for production of extruded rod stock for machining of for use in injection molding. Printers commercially available from Intamsys are capable of extrusion printing of PEEK polymer.

PEEK does not bond well to tissue and both PEEK and PEKK materials are susceptible to microbial contamination and to the support of bacterial biofilms. Composites of zeolite with PEEK produce a more hydrophilic and negatively charged surface which is less favorable to bacterial adhesion and more receptive to tissue attachment and integration. The hydrophilicity imparted by the zeolite results in an engineered biomaterial that interacts with the bone of the patient and induces a bone/biomaterial fusion. The presence of the zeolite also results in a rapid transition from M1 proinflammatory macrophage phenotype to the M2 macrophage phenotype, thereby minimizing fibrous encapsulation and facilitating the deposition of cite appropriate tissue ultimately yielding constructive and functional tissue remodeling. The negative charge imparted by the zeolite attracts and adheres the required precursor proteins for bone growth to the implant surface, and ultimately supports long term osseointegration.

Furthermore, zeolite incorporated into the polymer and exposed at the surface can be post-loaded (e.g., at temperatures between 0-100° C., preferably room temperature) with therapeutic metal ions such as silver, zinc, copper, strontium, magnesium etc. These materials will strongly inhibit attachment of microorganisms and can accelerate healing and reduce inflammation. By loading antimicrobial metal ions at these temperatures, deleterious oxidation of the metal ions that occurs at higher processing temperatures is reduced or eliminated.

In some embodiments, either natural zeolites or synthetic zeolites may be used to make the zeolites used in the embodiments disclosed herein. “Zeolite” is an aluminosilicate having a three-dimensional skeletal structure that is represented by the formula: XM_{2/n}O.Al₂O₃.YsiO₂.ZH₂O, wherein M represents an ion-exchangeable ion, generally a monovalent or divalent metal ion, n represents the atomic valency of the (metal) ion, X and Y represent coefficients of metal oxide and silica respectively, and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. A-type zeolites are particularly preferred, such as 4A zeolite having particle size ranges from 1 to 10 microns with a narrow distribution of about 4 microns.

Other ceramics and metal glasses are also envisaged instead of zeolite and are within the scope of the embodiments disclosed herein. For example, zirconium phosphate or silver glass could be used.

In certain embodiments, fine zeolite powder may be incorporated into a powder of the thermoplastic polymer. For example, 4 micron powder of a 4A Zeolite may be incorporated into PEEK powder that has a particle diameter of between about 10 to about 100 microns. In some embodiments, the incorporation of the zeolite into the polymer is carried out by thorough mixing the dry components at room temperature until the resulting composition is uniform by visual inspection. In some embodiments a drum roller can be used to carry out the mixing process.

The powder formulation may include the polymer, such as PEEK, and metal-loaded zeolite, such as silver zeolite.

In certain embodiments, when metal cation is used, the metal cation is present at a level below the ion-exchange capacity in at least a portion of the zeolite particles. In some embodiments, the amount of zeolite mixed with the polymer may range from about 5 to 50 wt. %, more preferably about 10 to 20 wt. %. The amount of metal ions, if present, in the zeolite should be sufficient such that they are present in an antimicrobial effective amount when implanted into the body of a patient. For example, suitable amounts can range from about 0.1 to about 20 or 30% of the exposed zeolite (w/w %). These levels can be determined by complete extraction and determination of metal ion concentration in the extraction solution by atomic absorption. Preferably the ion-exchanged antimicrobial metal cations, if present, are present at a level less than the ion-exchange capacity of the ceramic particles. The amount of ammonium ions is preferably limited to from about 0.5 to about 15 wt. %, more preferably 1.5 to 5 wt. % For applications where strength is not of the utmost importance the loading of zeolite can be taken as high as 50%. At such loadings the permeation of metal ions can permeate well below the surface layer due to interparticle contact, and much greater loadings of metal ions are possible.

In some embodiments, the powder formulation can be formed by adding zeolite that is devoid of metal ions, and then zeolite in the printed device can be post-loaded with metal ions. Metal ion salt solutions, such as nitrates, acetates, benzoates, carbonates, oxides, etc., can be used to accomplish this. Addition of nitric acid to the infusion solution also may be advantageous in that it can etch the surface of the implant, providing additional surface area for ion exchange. That is, the zeolite may be charged with metal ions at a temperature between about 0 and 100° C., preferably about room temperature) from a metal ion source such as an aqueous metal ion solution, such as silver nitrate, copper nitrate and zinc nitrate, alone or in combination. Cooling to lower temperatures gives lower loading rates but higher stability. Loading at even higher temperatures can be carried out at a faster rate by maintaining the system under pressure, such as in a pressure cooker or autoclave. The content of the ions can be controlled by adjusting the concentration of each ion species (or salt) in the solution.

For example, the printed PEEK zeolite composite can be loaded by bringing the material into contact with an aqueous mixed solution containing ammonium ions and antimicrobial metal ions such as silver copper, zinc etc. The most suitable temperatures at which the infusion can be carried out range from 5° C. to 75° C., but higher temperatures may also be used even above 100° C. if the reaction vessel is held under pressure. Higher temperatures will show increased infusion rates, but lower temperatures may eventually produce more uniform and higher loadings. The pH of the infusion solution can range from about 2 to about 11 but is preferably from about 4 to about 7. Suitable sources of ammonium ions include ammonium nitrate, ammonium sulfate and ammonium acetate. Suitable sources of the antimicrobial metal ions include: a silver ion source such as silver nitrate, silver sulfate, silver perchlorate, silver acetate, diamine silver nitrate and diamine silver nitrate; a copper ion source such as copper(II) nitrate, copper sulfate, copper perchlorate, copper acetate, tetracyan copper potassium; a zinc ion source such as zinc(II) nitrate, zinc sulfate, zinc perchlorate, zinc acetate and zinc thiocyanate.

In certain embodiments, control of the porosity of the final product can be carried out by the addition of a porogen, such as a salt. Complete porosity can be expected at 50 (volume/volume) %. For non-load bearing surfaces up to 50% salt may be used, producing an open cell structure which is completely permeable and accessible to adjacent tissue and fluids. Furthermore, the porosity allows the delivery of therapeutic agents from throughout the structure of the device and allows bone and tissue to grow deep into and through the device structure. In certain embodiments, the soluble salts are thoroughly and completely washed from the matrix with pure water. In some embodiments, suitable porogens include sodium or potassium chloride, calcium phosphate, sulfate or silicate, sodium citrate, sodium tartrate, ammonium bicarbonate, ammonium chloride, sodium fluoride, potassium fluoride, sodium iodide, sodium nitrate, sodium sulphate, sodium iodate, and mixtures thereof. Residual exposed salt can be washed from the surface of the implant using pure water. Preferably the salts are used in fine powder form and are water soluble so that they easily can be removed from the device such as after the device is cooled. Preferably the porogen is micronized salt, preferably sodium chloride, having an average particle size ranging from 2 to 10 microns, more preferably from 4 to 8 microns. In some embodiments, the size of the porogen is similar to or about the same as the size of the zeolite particles. Micronizing the porogen allows for the powder mixture to remain homogeneous through the powder handling steps of the process, and allows uniform pore distribution in the resulting composite. Suitable amounts of porogen include from about 2 to about 50% by weight of the composite blend, more preferably from about 5 to about 20% by weight. The result is a tortuous path within the pore network that will result in a much-enhanced capability for the device to carry exchanged ions as well as providing more prolonged release kinetics.

The entire device may be made porous in this way, or the device may have a porosity gradient, with the highest porosity at or near the surface which may facilitate attachment of developing bone and tissue to the implant surface once the device is implanted in a host patient. In certain embodiments, the device may be formed to have one or more solid regions and one or more porous regions. Since the 3D process forms the implant layer by layer, it may be advantageous to use this feature to deposit layers with different amounts of zeolite and/or different amounts of porogen. For example, powder that includes zeolite could be used only for the layers at and near the outer surface to provide the hydrophilicity, but pure PEEK powder could be used internally.

In some embodiments, the mechanical strength of the device may be reinforced by incorporating carbon fiber into the powder formulation. For example, milled carbon fiber may be added to the powder mixture of zeolite and polymer, and the resulting mixture then subjected to the additive manufacturing process as set forth above. The carbon fiber may also result in greater inter-layer adhesion and integrity of the device. The incorporation of fibers or other suitable reinforcing material(s) provides high wear resistance, a Young's modulus of 12 GPa (matching the modulus of cortical bone) and providing sufficient strength to permit its use in very thin implant designs which distribute the stress more efficiently to the bone. The amount of reinforcing material such as carbon fiber incorporated into the resin such as PEEK can be varied, such as to modify the Young's modulus and flexural strength. One suitable amount is 30 wt % carbon fiber.

The resulting device may be introduced into the body surgically. Suitable hosts include mammals, including humans, canines, felines, livestock, primates, etc. The rate of release of antimicrobial metal ions, if present, is governed by the extent of loading of the polymer with zeolite and the extent to which the exposed zeolite is charged with metal ions. The electrolyte concentration in host blood and body fluids is relatively constant and will cause ion exchange with ions such as silver, copper and zinc, etc. from the surface of the implant, which deactivate or kill gram positive and gram negative organisms, including E. coli and Staphylococcus aureus. Effective antimicrobial control (e.g., a six log reduction of microorganisms) is achieved even at low metal ion concentrations of 40 ppb.

### Formulation Example 1

| | Composition | Average particle |
|---|---|---|
| Component | (w/w) % | size (Microns) |
| PEEK | 88 | 60 |
| 4A Zeolite | 12 | 4 |

### Formulation Example 2

| | Composition | Average particle |
|---|---|---|
| Component | (w/w) % | size (Microns) |
| PEEK | 88 | 10 |
| 4A Zeolite | 12 | 4 |

### Formulation Example 3

| | Composition | Average particle |
|---|---|---|
| Component | (w/w) % | size (Microns) |
| PEEK | 73 | 10 |
| 4A Zeolite | 12 | 4 |
| Carbon Fiber | 15 | Milled strand |

### Formulation Example 4

| | Composition | Average particle |
|---|---|---|
| Component | (w/w) % | size (Microns) |
| PEEK | 73 | 10 |
| 4A Zeolite | 12 | 4 |
| Carbon Fiber | 15 | Milled strand |

### Formulation Example 5

| | Composition | Average particle |
|---|---|---|
| Component | (w/w) % | size (Microns) |
| PEEK | 43 | 10 |
| Sodium Chloride | 3 | 4 |
| 4A Zeolite | 12 | 4 |
| Carbon Fiber | 15 | Milled strand |

### Example 6

PEEK powder, available from Solvay, about 10 micron particle size diameter, is carefully weighed out. Silver zeolite, 4A Zeolite loaded to 22-24% silver, of about 4 micron particle size, is added to the PEEK powder in a container in the amount shown in the Formulation Examples, and both materials are mixed thoroughly by rotating the container on a drum roller for about 10 minutes.

The resulting composite mixture is added to a machine such as the ESOINT P 800 3D printer. A 3D CAD diagram of the desired part or object may be drawn or generated by scanning the object to be duplicated. The 3D data is sliced into thin layers and may be the input data for the selective laser sintering machine. A CO₂ laser controlled by the CAD data selectively scans and fuses the powder in the base layer generating a first slice of the object. A fresh layer of powder, such as a layer 1 mm thick, may be spread over the surface of the first fused layer and powder bed and the data from the second CAD slice may be used to fuse the second layer on top of the first, and so on. When the object is fully formed, it may be removed from the powder bed and brushed free of the loose powder.

In the case of fusing high temperature materials, the temperature of the powder in the bed may be raised to just below the melting point of the powder and the laser merely supplies sufficient energy to raise the temperature above the melting point, allowing the material targeted by the laser to melt and fuse.

While various aspects and embodiments have been disclosed herein, other aspects, embodiments, modifications and alterations will be apparent to those skilled in the art upon reading and understanding the preceding detailed description. The various aspects and embodiments disclosed herein are for purposes of illustration and are not intended to be limiting. It is intended that the present disclosure be construed as including all such aspects, embodiments, modifications and alterations insofar as they come within the scope of the appended claims or the equivalents thereof.

What is claimed is:

1. A surgically implanted device implanted and osseointegrated in a patient at an implant site, said device having a surface exposed to bodily fluids of said patient and comprising PEEK and zeolite incorporated in said PEEK in an amount sufficient to impart a negative charge to said exposed surface, said implanted and osseointegrated device upon implantation having caused rapid transition from M1 proinflammatory macrophage phenotype to M2 macrophage phenotype at said implant site in said patient, effective to minimize fibrous encapsulation of said device and yield constructive bone fusion to said device to osseointegrate said device in said patient.

2. The surgically implanted device of claim 1, wherein said device is configured as a screw.

3. The surgically implanted device of claim 1, wherein said device is configured as a spinal implant.

4. The surgically implanted device of claim 1, wherein said device is a configured as a dental implant.

5. The surgically implanted device of claim 1, wherein said device is a configured as a hip stem.

6. The surgically implanted device of claim 1, wherein said device is configured as a skull flap.

7. The surgically implanted device of claim 1, wherein said device is configured as a trauma plate.

8. The surgically implanted device of claim 1, wherein said device is configured as a knee implant.

9. The surgically implanted device of claim 1, wherein said device is radiotranslucent.

10. A surgically implanted tissue integrated device implanted in a patient at an implant site, said device having a surface exposed to bodily fluids of said patient and comprising PEEK and zeolite incorporated in said PEEK in an amount sufficient to impart a negative charge to said exposed surface, said implanted and tissue integrated device upon implantation having caused rapid transition from M1 proinflammatory macrophage phenotype to M2 macrophage phenotype at said implant site, effective to minimize fibrous encapsulation of said device and facilitate the deposition of said integrated tissue yielding constructive tissue remodeling to integrate said tissue and said device in said patient.

11. The surgically implanted device of claim 10, wherein said device is configured as a screw.

12. The surgically implanted device of claim 10, wherein said device is configured as a spinal implant.

13. The surgically implanted device of claim 10, wherein said device is configured as a dental implant.

14. The surgically implanted device of claim 10, wherein said device is configured as a hip stem.

15. The surgically implanted device of claim 10, wherein said device is configured as a skull flap.

16. The surgically implanted device of claim 10, wherein said device is configured as a trauma plate.

17. The surgically implanted device of claim 10, wherein said device is configured as a knee implant.

18. The surgically implanted device of claim 10, wherein said device is radiotranslucent.
